(19) United States
(12) Patent Application Publication
(10) Pub. No.: US 2016/0166736 A1
(43) Pub. Date: Jun. 16, 2016

(54) OSTEOINDUCTIVE MATERIALS

(71) Applicant: THE UNIVERSITY OF LIVERPOOL, Liverpool (GB)

(72) Inventors: Judith Curran, Liverpool (GB); John Hunt, Liverpool (GB)

(73) Assignee: The University Of Liverpool, Liverpool (GB)

(21) Appl. No.: 14/908,919

(22) PCT Filed: Jul. 29, 2014
(86) PCT No.: PCT/GB2014/052322

§ 371 Date: Jan. 29, 2016

(30) Foreign Application Priority Data
Jul. 29, 2013 (GB) 1313495.2

(51) Int. Cl. A61L 27/54; C12N 5/0775; A61L 27/10; A61L 27/18; A61K 31/131; A61K 31/695

(52) U.S. Cl. CPC A61L 27/54; A61K 31/131; A61K 31/695; A61L 27/10; A61L 27/18; C12N 5/0662; A61L 2300/414; A61L 2430/02; A61L 2430/12; C12N 2533/12; C12N 2533/40; C12N 2533/00

(57) ABSTRACT

Provided is an osteoinductive material comprising a substrate; and a layer of a hydrocarbyl group comprising 10 to 12 carbon atoms, the hydrocarbyl group having one end attached to the substrate and a presenting amine (—NH2) group at the unattached end. The hydrocarbyl group may be 11 carbons long, and may be provided in the form of 11-aminoundecyltriethoxysilane. The substrate may be a glass; metal; polymer; ceramic; plastic; or hydroxyapatite. The substrate may, for example, be a metal selected from the group consisting of: titanium; titanium alloys; cobalt chrome; and steel. An osteoinductive material may be provided in the form of a medical implant, or part thereof, such as an orthopaedic implant or a dental implant or part thereof. Alternatively the substrate may be in the form of a cell culture vessel. Also provided is a method of producing such osteoinductive materials, the medical use of such materials, and a method of producing bone or a bone precursor utilising the disclosed materials.

The present invention relates to osteoinductive materials, and to methods for the production of such osteoinductive materials. The invention also relates to the use of the disclosed osteoinductive materials for the production of bone, and to methods of producing bone or bone precursor using such osteoinductive materials. A further aspect of the invention relates to medical uses of the osteoinductive materials.

Traditional approaches to the differentiation of cells ex vivo have tended to use naturally occurring biologically active substances such as growth factors to direct the differentiation of cells. Such growth factors, recombinant forms of which may typically be used in cell culture applications, tend to be costly. They are also frequently subject to degradation over time, which leads to them having short shelf lives.

There is an increasing recognition of the importance of the way in which materials can influence the activities of biological cells that are in contact with them, and an aim to develop materials that can influence these activities in a way that produces a desired effect.

For example it has been found that materials functionalised by the presence of —NH₂ groups attached to short hydrocarbon chains (three carbon chain length) are able to cause changes in mesenchymal stem cell gene expression profiles that are indicative of osteogenic differentiation.

Accordingly, there is growing interest in the use of biologically active materials in cell culture applications to direct the differentiation of cells in a manner that allows ex vivo generation of desired cell types or tissues.

Interest in such biologically active materials is not only limited to ex vivo applications. Medical implants represent a highly suitable context in which biologically active materials may be used to influence the activity of a patient's cells around the site of the implant.

The need for medical implants, such as orthopaedic implants, is expected to grow for the foreseeable future, as the aged population grows and expects to retain a high degree of mobility and quality of life. Some estimates suggest that the numbers of primary total hip and knee arthroplasty may grow respectively by 174% to 572,000 and by 673% to 3,480,000 procedures between 2005 and 2030 in the US.

Despite high clinical success rates associated with the introduction of orthopaedic implants, problems can arise that ultimately compromise the success of the implant in situ. Among these, one of the most common failings is insufficient integration between the material of the implant and the patient's tissue. This can cause loosening of the implant which may require corrective surgery or removal of the implant in order to be addressed.

Titanium is commonly used in the manufacture of implants, and exhibits excellent properties as an orthopaedic biomaterial. However, titanium implants are not osteogenic or osteoinductive. In an attempt to address this failing, numerous studies have investigated the modification of the titanium surface in order to enhance the implants bioactivity and hence optimise the bone-to-implant contact to provide complete integration and intimate bone apposition. Modification strategies reported in the prior art include blasting the alkaline surface with bioactive media, acid or alkaline etching, anodisation, and surface immobilisation of specific biofunctional molecules.

A common commercialised technique used for clinical orthopaedic and dental applications is plasma spraying of a (bioactive) calcium phosphate based coating, such as hydroxyapatite, onto the titanium surface to mimic the inorganic components of bone tissue. These techniques have achieved a certain level of success, and the surface-modified implants have been used clinically. However, the speed of bony ingrowth can be slow, delaying the patient's ability to fully utilise the implant.

Hydroxyapatite currently represents the “gold standard” for coating orthopaedic devices, and there is much research and development focused around methods of deposition of the hydroxyapatite coating, as well as suitable variations of hydroxyapatite coatings.

In view of the above it can be seen that there remains a need for new osteoinductive materials. The present invention relates to osteoinductive materials, and to methods for the production of such osteoinductive materials. The invention also relates to the use of the disclosed osteoinductive materials for the production of bone, and to methods of producing bone or bone precursor using such osteoinductive materials. A further aspect of the invention relates to medical uses of the osteoinductive materials.

In a first aspect the present invention provides an osteoinductive material comprising:
a substrate; and
a layer of a hydrocarbyl group comprising 10 to 12 carbon atoms, the hydrocarbyl group having one end attached to the substrate and a presenting amine (—NH2) group at the unattached end.

An “osteoinductive” material in accordance with the present invention is one that induces cells with osteogenic potential to develop into the bone-forming lineage. In suitable embodiments the cells with osteogenic potential may be stem cells. Examples of suitable stem cells are described elsewhere in this application, and include mesenchymal stem cells.

Suitable hydrocarbyl groups for use in the osteoinductive materials of the invention may be straight or branched. In a preferred embodiment the hydrocarbyl group is straight.

In a suitable embodiment the hydrocarbyl group is a 10 to 12 carbon alkyl chain, and in a particularly suitable embodiment that hydrocarbyl group is an 11 carbon alkyl chain.

In a suitable embodiment, the hydrocarbyl group referred to in connection with the various aspects of the invention is substantially the only hydrocarbon present on a coated surface of an osteoinductive material of the invention. In a suitable embodiment, the hydrocarbyl group is substantially the only hydrocarbon present on an osteoinductive material of the invention.

In a suitable embodiment the hydrocarbyl group is attached to the substrate via a Si—O—Si bond.

The substrate employed in an osteoinductive material of the invention may comprise one or more constituents selected from the group consisting of: glass, metals; polymers; ceramics; and plastics. Suitable substrates may be determined with reference to the application in which the osteoinductive material is to be used, as discussed further below.

For the purpose of the present disclosure, except for where the context requires otherwise references to “metals” shall be taken as encompassing metal alloys. Thus, by way of example, osteoinductive materials according to the invention may employ a substrate comprising a metal selected from the group consisting of: steel; titanium; titanium alloys; and cobalt chrome.

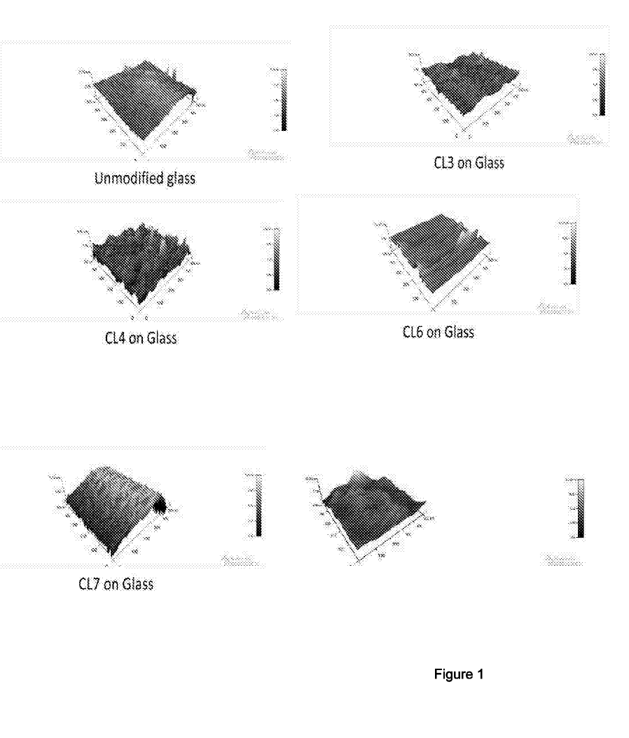

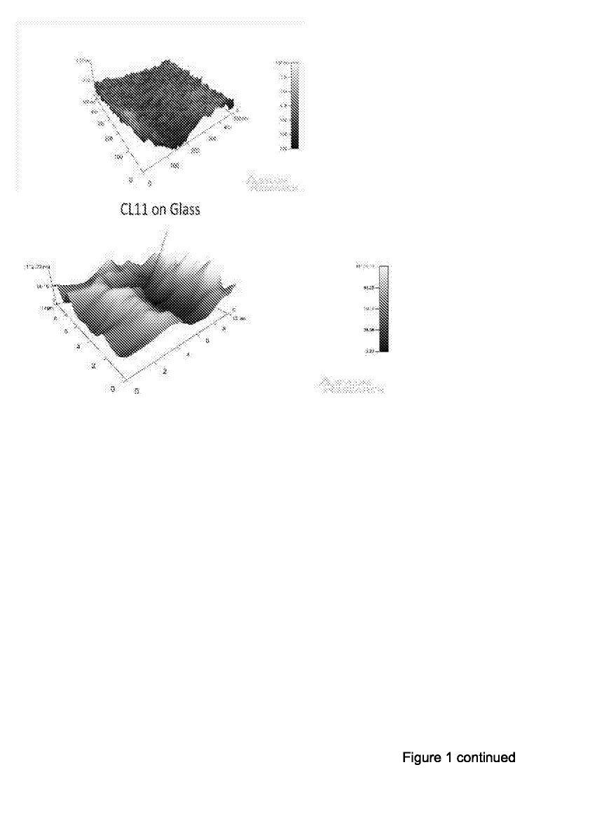

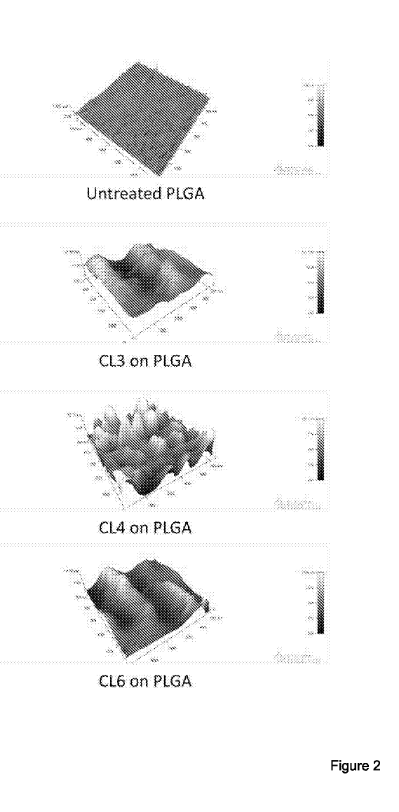

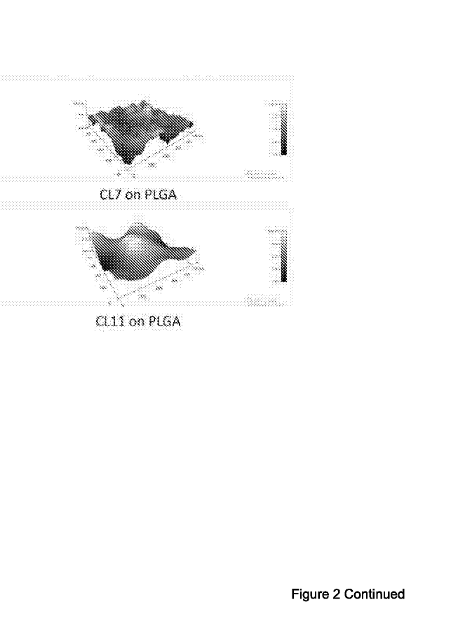

The materials of the invention preferably comprise multiple layers of the hydrocarbyl group and presenting amine. These multiple layers tend to be distributed unevenly over the surface of the material, so that different depths of the layers occur over different regions of the material's surface. As a result, the multiple layers give rise to a number of nanometre-scale features on the surface of the osteoinductive materials of the invention. These features can be seen in the Figures described elsewhere in the specification (in particular in FIGS. 1 and 2) and have a height range of between approximately 50 and 155 nm (in particular materials of the invention utilising a polymer substrate may be characterised by the presence of nanometre-scale features having a height of between 50 nm and 155 nm; while materials of the invention utilising a glass substrate may be characterised by the presence of nanometre-scale features having a height of between 115 nm and 155 nm). This contrasts with the maximum height of nanometre-scale features found in comparison materials (produced using hydrocarbyl groups having chain lengths outside those specified in respect of the materials of the invention) which demonstrate a maximum height in the region of 35 nm.

Atomic force microscopy images of the surfaces of the materials of the invention indicate that the nanometre-scale features overlie a micrometer-scale topography.

The arrangement of the features on the surface of the material may also prove to be characteristic of the osteogenic materials of the invention. The features have an “open honeycomb” arrangement, as can be seen from microscopic images of the materials. This arrangement is also observed in the extracellular matrix components that biological cells deposit when cultured on the materials of the invention.

Without wishing to be bound by any hypothesis, the inventors believe that this “topography” of the surfaces of the osteoinductive materials of the invention, optionally in combination with features of the surface energy and surface chemistry of the materials described elsewhere in the specification, controls adsorption of proteins to the surface, which makes an important contribution to the biological activities exerted by the osteoinductive materials of the invention. This adsorption of proteins may be selective, in that it appears to particularly favour the adsorption of certain proteins, including (but not limited to) proteins selected from the group consisting of: fibronectin; and vitronectin.

In a suitable embodiment, an osteoinductive material of the invention has a maximum “feature height” at of least 40 nm.

In an alternative embodiment, an osteoinductive material of the invention has a maximum feature height of at least 60 nm.

In a still further embodiment, an osteoinductive material of the invention has a maximum feature height of at least 90 nm.

In a suitable embodiment, an osteoinductive material of the invention has a maximum feature height is between approximately 50 and 155 nm. This corresponds to a depth of the hydrocarbyl group on the surface of the osteoinductive material that ranges between approximately 50 and 155 nm.

In a suitable embodiment, an osteoinductive material of the invention has a maximum feature height is between approximately 90 and 150 nm. This corresponds to a depth of the hydrocarbyl group on the surface of the osteoinductive material that ranges between approximately 90 and 150 nm.

In suitable embodiments of the materials of the invention in which the substrate comprises a polymer, the maximum feature height may be approximately 100 nm. This corresponds to a maximum depth of approximately 100 nm of the hydrocarbyl group on the surface of the osteoinductive material.

Alternatively, embodiments of materials of the invention in which the substrate comprises a polymer may have maximum feature height of approximately 140 nm. This corresponds to a maximum depth of approximately 140 nm of the hydrocarbyl group on the surface of the osteoinductive material.

As referred to above, the inventors believe that the surface energies of the osteoinductive materials of the invention have an important impact (optionally in combination with the topography the materials' surfaces) upon the effects that they are able to exert upon biological cells. In a suitable embodiment an osteoinductive material of the invention may have a water contact angle at the surface of the material is less than 100°.

Suitably, an osteoinductive material of the invention may have a water contact angle at the surface of the material of between approximately 85° and 95°. In a particularly suitable embodiment the water contact angle at the surface of the material is approximately 90°.

These considerations regarding surface energy and water contact angles may be of particular relevance to embodiments of the osteogenic materials of the invention in which the substrate comprises a polymer.

In a second aspect, the invention provides a method of producing an osteoinductive material, the method comprising contacting a surface of a substrate with a solution containing a compound comprising a hydrocarbyl group comprising 10 to 12 carbon atoms and a terminal amine group, such that a coating of the hydrocarbyl group is formed on at least a portion of a surface of the substrate.

Suitable hydrocarbyl groups for use in these methods of the invention may be straight or branched. In a preferred embodiment the hydrocarbyl group used in the method is straight.

The hydrocarbyl group may be a 10 to 12 carbon alkyl chain, and in a preferred embodiment that hydrocarbyl group is an 11 carbon alkyl chain. In a particularly preferred embodiment the hydrocarbyl group is provided in the form of 11-aminoundecyltriethoxysilane.

In a suitable embodiment the method of the invention may be one, wherein the solution contains the hydrocarbyl group-containing compound (such as 11-aminoundecyltriethoxysilane) at a concentration of approximately 0.1 M. The inventors have found that methods in accordance with this embodiment give rise to osteodinductive materials that induce a response in cells that is homogenous across the surface that has been contacted with the hydrocarbyl group. It will be appreciated that this homogeneity of response confers advantages when the osteoinductive material is used, and so such homogenous materials will generally be viewed as being of increased quality. Without wishing to be bound by any hypothesis, the inventors believe that this property may be associated with a homogenous distribution of amine groups across the coated surface of the material. The distribution of amine groups can be determined using the ninhydrin assay as described elsewhere in the specification.

The methods of the invention may make use of any of the substrates considered elsewhere in the specification, including glass; metals; polymers; ceramics; and plastics.

Accordingly, the substrate may comprise a metal selected from the group consisting of: steel; titanium; titanium alloys; and cobalt chrome.

In a suitable embodiment the substrate may be in the form of an item that it is wished to provide with osteoinductive properties. For example, the substrate may be provided in the form of a medical implant (or part thereof) or a cell culture vessel (or part thereof).

A suitable manner in which the surface of the substrate may be contacted with the solution containing the hydrocarbyl group is by immersion of the substrate within the solution. In an alternative embodiment, the surface of the substrate may contacted with the solution by spraying the solution onto the surface of the substrate

Once the coating of the hydrocarbyl group has been formed, the method may further comprise washing the coated substrate in alcohol and/or water. The method of producing an osteoinductive material may further comprise drying the surface of the substrate.

For the purposes of the present disclosure, either an osteoinductive material in accordance with the first aspect of the invention, or an osteoinductive material produced by the methods of the second aspect of the invention, may be referred to as “an osteodinductive material of the invention”. The osteoinductive materials produced by the methods of the second aspect of the invention may have any of the features described with reference to the first aspect of the invention.

In a suitable embodiment, an osteoinductive material of the present invention may be provided in the form of a medical implant, or a part thereof, such as those provided to a patient during surgery. Suitably an osteoinductive material of the invention in accordance with this embodiment may be in the form of an orthopaedic implant (or a part thereof). Alternatively an osteoinductive material in accordance with this embodiment of the invention may be in the form of a dental implant (or a part thereof).

When osteoinductive materials of the invention in the form of a medical implant are provided to a patient, they are suitable for use for the production of bone in vivo in accordance with the second aspect of the invention.

When an osteoinductive material of the invention is provided in the form of a medical implant, such as those described above, the hydrocarbon layer is preferably provided over some or all of the surface on which it is desired that bone should be produced. Merely by way of example, this may be a surface that is in contact with the patient's existing bone, in which case the production of new bone promoted by the osteogenic material of the invention may serve to integrate the implant with the existing bone, thus securing the implant.

An osteodinductive material of the invention may be provided in the form of a cell culture vessel, or a part thereof. Various forms of cell culture vessel are known to those skilled the art, including (but not limited to): cell culture dishes (such as Petri dishes); cell culture flasks; cell culture plates; cell culture bottles (such as roller bottles); cell culture bags; and cell culture beads.

Cell culture vessels in accordance with this embodiment may utilise as substrates any suitable material that is conventional in the manufacture of such vessels. Typically the material from which a cell culture vessel is manufactured will be a non-toxic, non-pyrogenic material. A suitable substrate for use in such cell culture materials may be selected from the group consisting of: glass; polystyrene; poly(lactic-co-glycolic acid) PLGA; poly(ether)urethanes; polyesters; and polycaprolactone.

When an osteoinductive material of the invention is provided in the form of a cell culture vessel, such as those described above, the hydrocarbon layer is preferably provided over some or all of the surface on which cells are grown. Cell culture vessels comprising an osteoinductive material of the invention are particularly suitable for use in the uses of the invention, in which bone is produced ex vivo, such as in vitro.

In the event that the substrate is in the form of a part of a cell culture vessel, this part may be a portion that in use will come into contact with cells to be cultured.

In a third aspect, the invention provides the use of an osteoinductive material of the invention for the production of bone or a bone precursor. In the context of the present disclosure “a bone precursor” may be taken as encompassing the extracellular matrix that is found prior to ossification. A bone precursor of this sort may be characterised by the presence of extracellular osteocalcin, and/or by the calcification of the extracellular matrix. For the sake of brevity, references to “bone” in the present specification should also be taken as encompassing “bone precursor”, except for where the context requires otherwise.

The production of bone in the context of the third aspect of the invention may be production of bone in vivo, or production of bone ex vivo, such as production of bone in vitro.

In a fourth aspect, the invention provides a method of producing bone or a bone precursor, the method comprising:
providing an osteoinductive material according to the invention;
providing a population of cells with osteogenic potential; and
maintaining the cells in contact with the material until bone or a bone precursor is produced.

Generally, in the uses and methods of the third and fourth aspects of the invention, bone will be produced if the use or method in question is continued after bone precursor has been formed. Thus the methods or uses may further comprise maintaining cells in contact with the material until the bone precursor is remodelled into bone or a bone-like substance.

It will be appreciated that these methods of the invention provide many of the same advantages that have already been described in connection with the osteoinductive materials of the invention. In particular the methods of the invention increase the consistency and reproducibility of the bone produced, since the materials exert an inherent osteoinductive effect upon the cells, rather than requiring the addition of supplements (the potency of which can vary significantly between batches) to induce bone production. The fact that the methods of the invention make it possible to avoid the need for use of cell culture supplements, such as growth factors, also provide economic advantages in that supplements of this sort are frequently very expensive.

As described elsewhere in the specifications, the materials of the invention have the ability to absorb calcium and phosphate onto their surfaces when placed in an environment that contains these substances. This absorption contributes to formation, by cells in contact with the material, of a very dense extracellular matrix on the surface of the material. As illustrated further in the Experimental Results section (in particular the results of x-ray analysis and von Kossa staining, images of which show very early positive staining), this effect is only observed in connection with materials of the invention (exemplified by experimental materials comprising an eleven carbon hydrocarbyly group) and not in other control materials (such as materials comprising hydrocarbyl groups with shorter carbon chain lengths).

The biological cells deposit and become encased in this extracellular matrix, and start to remodel the matrix, forming an even denser osteogenic matrix. It will be appreciated that the ability of the materials of the invention to stimulate the production of calcium/phosphate rich matrices that are highly osteoinductive has many useful applications. The methods by which these materials of the invention may be produced, using simple wet chemistry and adsorption, are significantly cheaper than other approaches using expensive hyaluronic acid (HA)/calcium phosphate (CaP) coating technologies.

In a suitable embodiment, the cells with osteogenic potential are stem cells. Merely by way of example, such stem cells may be selected from the group consisting of: embryonic stem cells; cord blood stem cells; adult stem cells (including haematopoietic stem cells; mesenchymal stem cells; and dental pulp stem cells); and induced pluripotent stem cells.

In a preferred embodiment of the methods of the invention, the stem cells are mesenchymal stem cells. Mesenchymal stem cells represent particularly suitable cells that may be induced to produce bone by the appropriate methods or uses of the invention, in that they have been shown to undergo strong osseoinduction in response to contact with the materials of the invention. Furthermore, such mesenchymal stem cells tend to be quite abundant at the sites where medical implants are inserted, since they are found at high numbers in the bone marrow and dental pulp. Furthermore, mesenchymal stem cells are considered very favourably as cells for use in ex vivo applications, since they can be obtained in relatively high numbers from sources that do not tend to cause ethical concerns (including sources such as cord blood or adipose tissue, as well as the bone marrow and dental sources mentioned above), are capable of expansion in culture, and have shown properties that make them suitable for transplantation.

In a suitable embodiment of the methods of the invention, the material of the invention and cells that are to be induced to form bone are provided with a source of phosphate and/or a source of calcium. In embodiments where the production of bone is taking place ex vivo, a suitable source of phosphate and/or calcium may be provided in a cell culture medium.

Suitably the cells and osteoinductive material are maintained in serum-free cell culture medium. Many existing protocols for the maintenance, expansion or differentiation of stem cells require the use of serum as a supplement to ensure cell growth. However, serum contains many factors that can have unwanted impact upon cells to which it is provided. Furthermore, since serum is derived from animal sources it is not suitable for use in the context of culturing cells that will be provided therapeutically, since there is the risk that the serum may contain infective agents that will contaminate the cells.

The ability of the materials of the invention to promote osteogenesis in serum-free culture conditions is surprising in that use of serum is a pre-requisite of many prior art techniques, and is also highly advantageous in that it enables the cultured cells to be used therapeutically avoids the problems of unwanted or unknown influence on cell activity.

In a suitable embodiment, a method of the invention may be one in which the cells are maintained in cell culture medium without osteogenic growth factors. It will be appreciated that the osteoinductive properties of the materials of the invention may render further osteogenic factors, such as grown factors, redundant. Indeed, in a suitable embodiment, a method of the invention may be one wherein the cells are maintained in cell culture medium substantially free of exogenous growth factors. Such methods provide advantages in that they avoid the costs associated with the addition of growth factors, such as osteogenic growth factors and also the unwanted variability that may arise when different concentrations or potencies of growth factors are used.

In a fifth aspect, the invention provides a method of promoting integration of a medical implant, the method comprising providing to a subject in need of such treatment a medical implant comprising an osteoinductive material of the invention.

It will be appreciated that the osteoinductive materials of the invention are suitable for use as medical implants in contexts where it is desirable for integration of the medical implant to be promoted. The ability of the osteoinductive materials of the invention to promote bone growth will be expected to provide advantages in terms of increases in the speed and extent of osteointegration that occur on introduction of the medical implant into its site in the patient. These increases may be expected to improve the effectiveness and longevity of such implants.

The invention will now be further described with reference to the following experimental results section, and the accompanying Figures, in which:

FIG. 1 shows atomic force microscopy (AFM) images of the surfaces of osteoinductive materials of the invention incorporating glass substrates. Changes in nanotopography associated with the different lengths of carbon chains presenting amine groups can clearly be observed. On chain lengths CL7 and CL11 maximum peak heights exceeded 10 nm and larger scale images were taken to assess the effect on microtopography as well as associated nanotopography. Images clearly show that chain lengths 7 and 11 resulted in a different surface nanotopography on top of a micro topography.

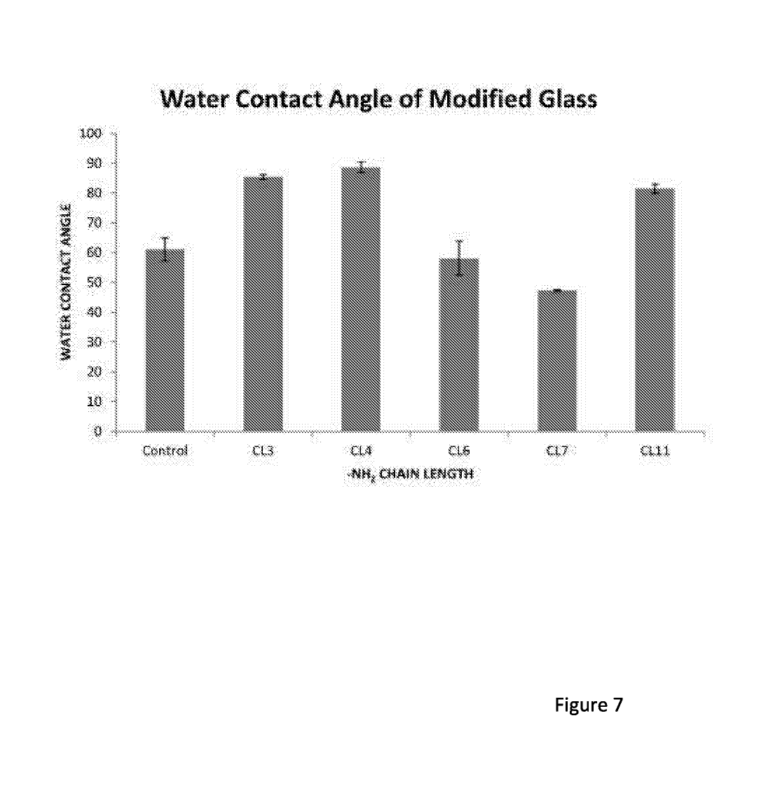

FIG. 2 shows atomic force microscopy (AFM) images of the surfaces of osteoinductive materials of the invention incorporating polymer (PLGA) substrates. Changes in nanotopography associated with the different lengths of carbon chains presenting amine groups can clearly be observed. Analysis of maximum peak heights is shown in FIG. 7.

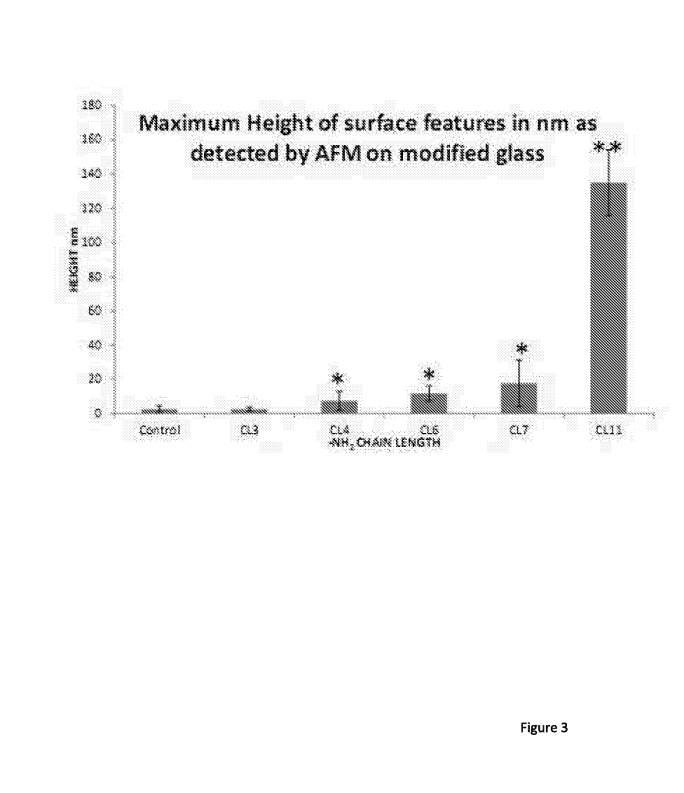

FIG. 3 is a graph showing quantitative analysis of maximum peak heights is provided in FIG. 1. Quantitative analysis of maximum height associated with different chain lengths was measured by AFM. Evaluation of the changes in height associated with the different chain lengths across entirety of the surface are shown in this Figure.

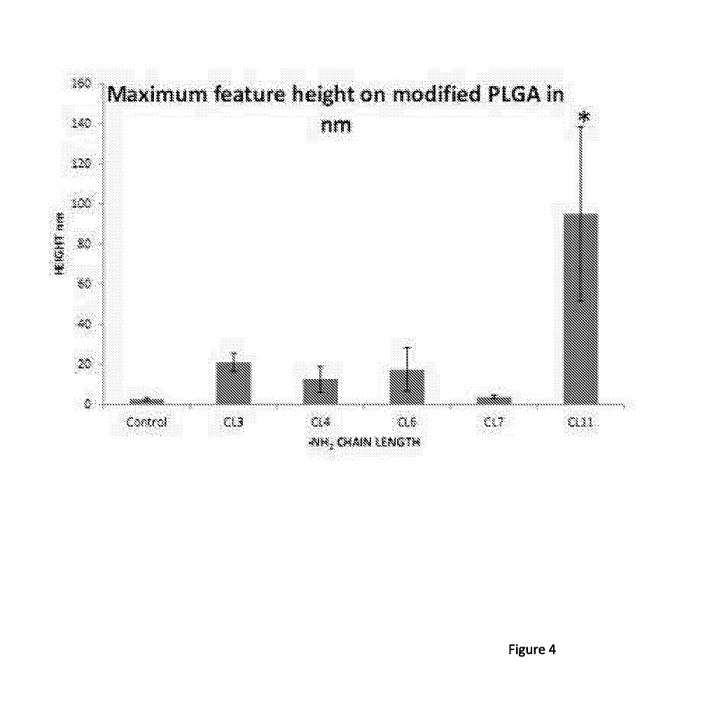

FIG. 4 is a graph showing quantitative analysis of maximum peak heights is provided in FIG. 2. Quantitative analysis of maximum height associated with different chain lengths was measured by AFM. Evaluation of the changes in height associated with the different chain lengths across entirety of the surface are shown in this Figure.

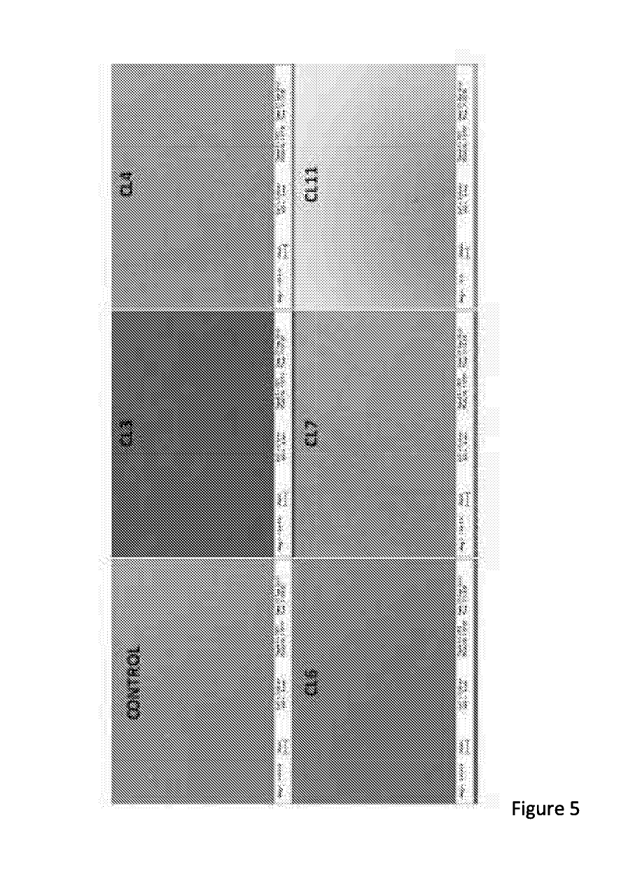

FIG. 5 shows scanning electron microscopy (SEM) images of film structure on materials (specifically, a control, experimental materials, and an osteoinductive material of the invention) incorporating a glass substrate. Nanometre-scale changes to the film structures are beyond the sensitivity of this technique, indications of gross changes in film topography associated with CL11, changes are proven by AFM analysis in FIG. 1.

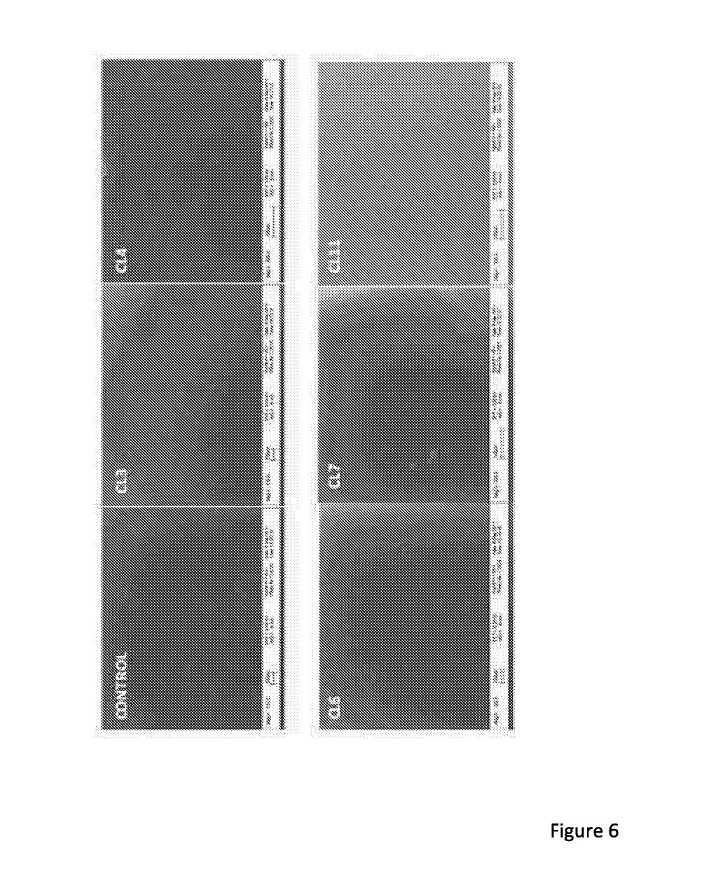

FIG. 6 shows scanning electron microscopy (SEM) images of film structure on materials (specifically, a control, experimental materials, and an osteoinductive material of the invention) incorporating a PLGA substrate. A change in film architecture can be observed with CL11 modifications (the osteoinductive material of the invention), a pattern that is repeated by matrix deposition associated with an enhanced osteogenic response induced by the material of the invention.

FIG. 7 is a graph showing water contact angle of the different chain length amines on glass substrates. Each chain length results in a specific change in associated surface energy as measured by water contact angle.

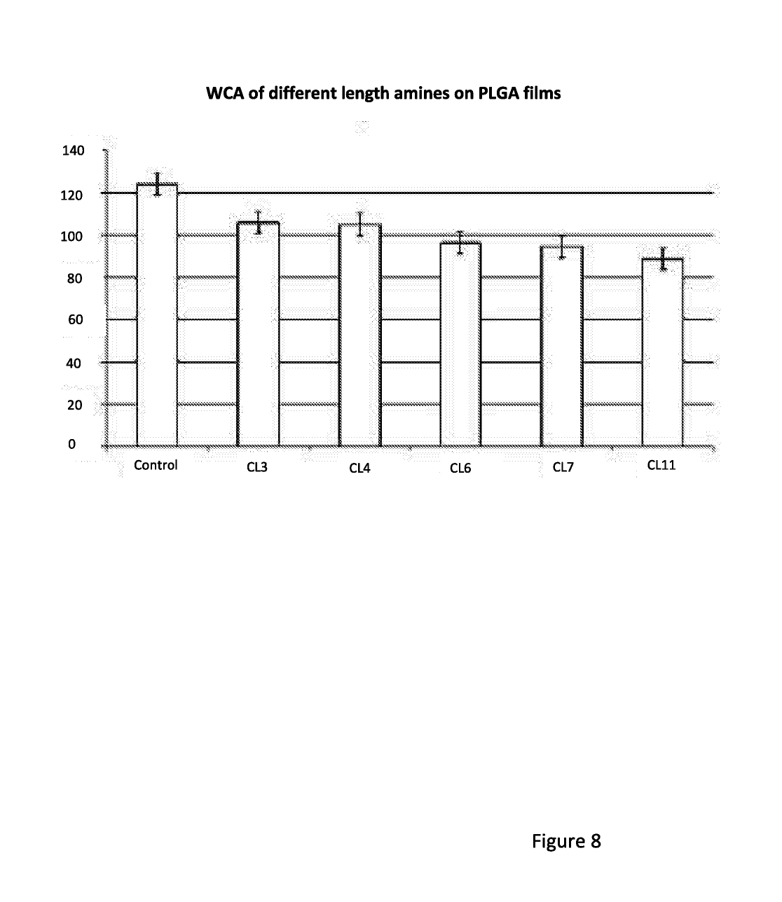

FIG. 8 is a graph showing water contact angle of the different chain length amines on a PLGA substrate. Amine modifications increase the hydrophilic nature of the PLGA film, each chain length results in a specific change of associated surface energy as measured by water contact angle. There is a specific surface energy, in the range 85-95, associated with the CL11 chain length.

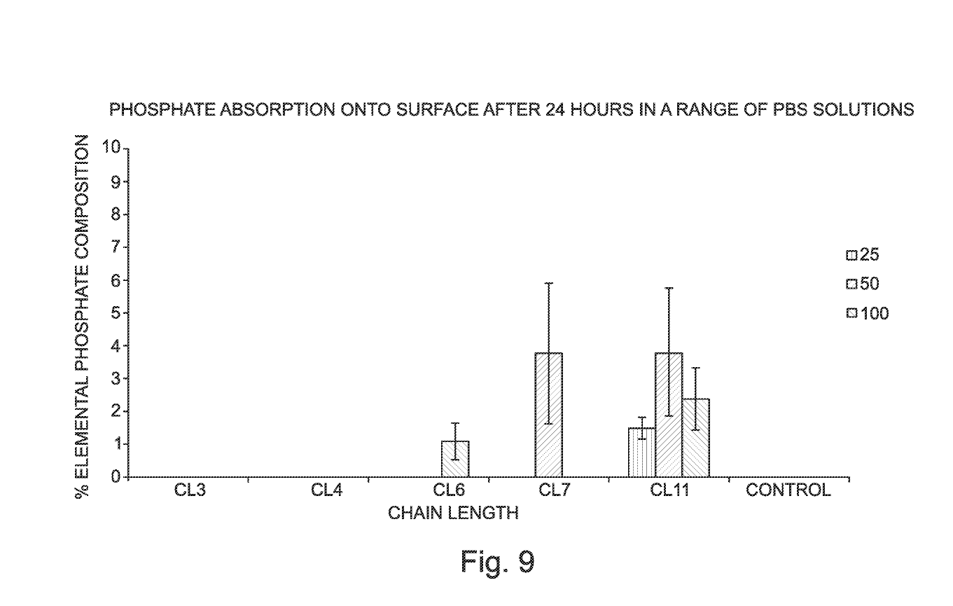

FIG. 9 is a graph comparing the results of X-ray analysis of phosphate absorption onto materials of the invention, and comparator materials, from various concentrations of phosphate buffered saline solution.

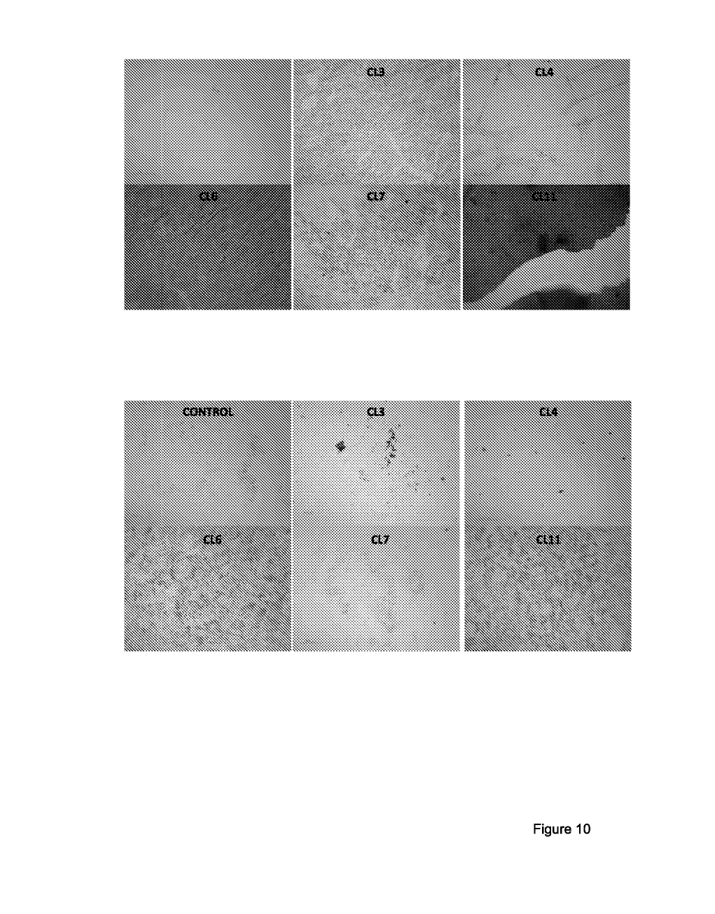

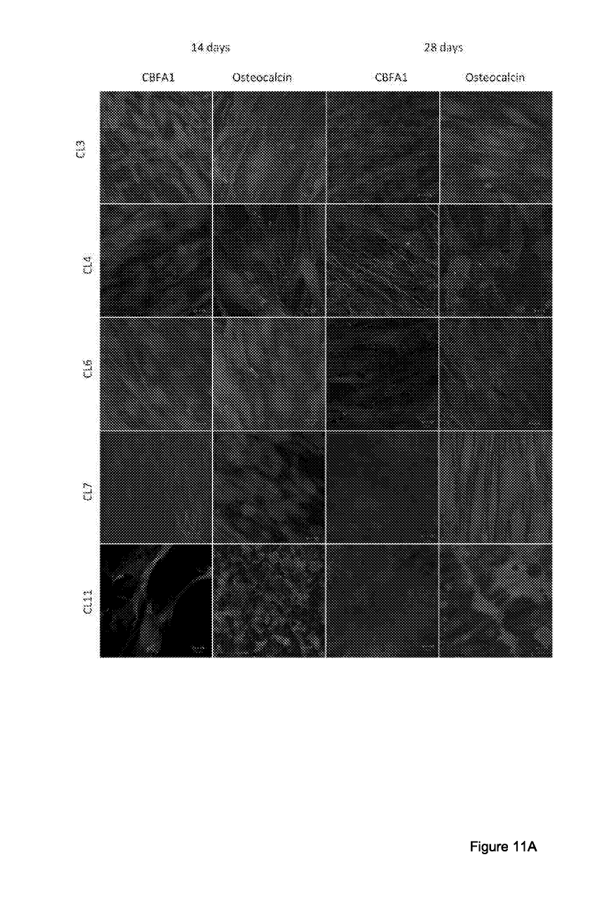

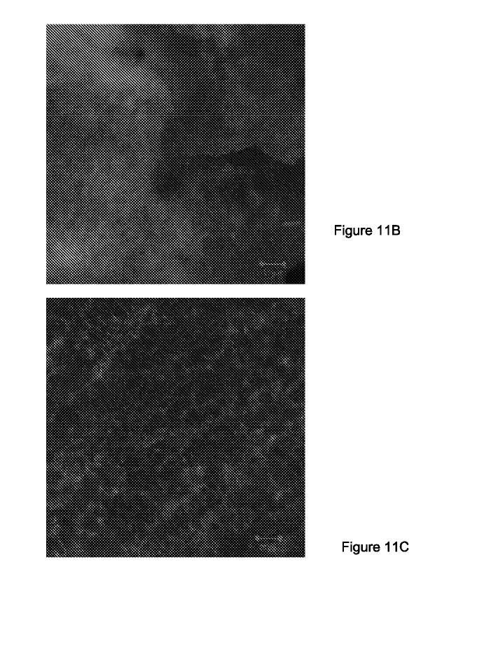

FIG. 10 shows micrographs of von Kossa staining of hMSC cultured on experimental materials utilising glass substrates for 7 (panel A) and 28 (panel B) days.

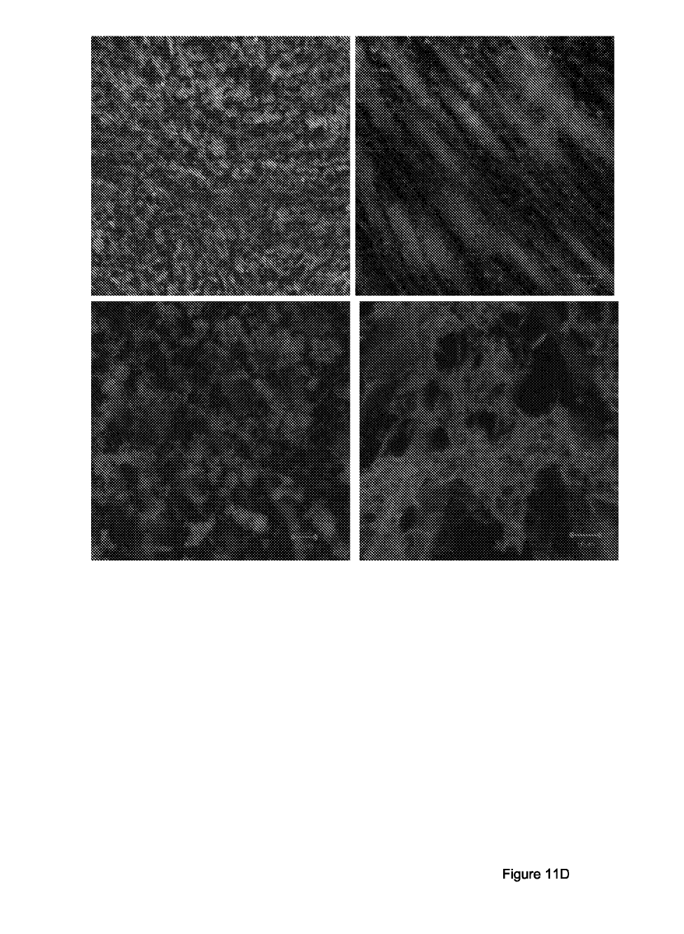

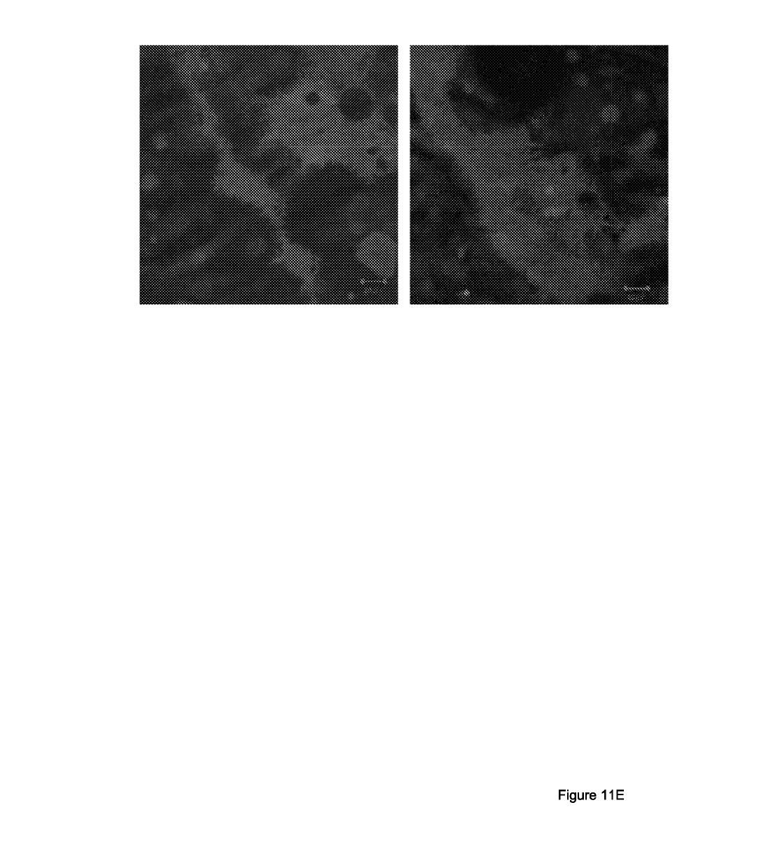

FIG. 11A-E fluorescent microscopy of hMSC on experimental materials utilising glass substrates at 14 or 28 days

FIG. 12 shows micrographs of von Kossa staining of hMSC cultured on experimental materials utilising polymer (PLGA) substrates for 7 (panel A) and 28 (panel B) days.

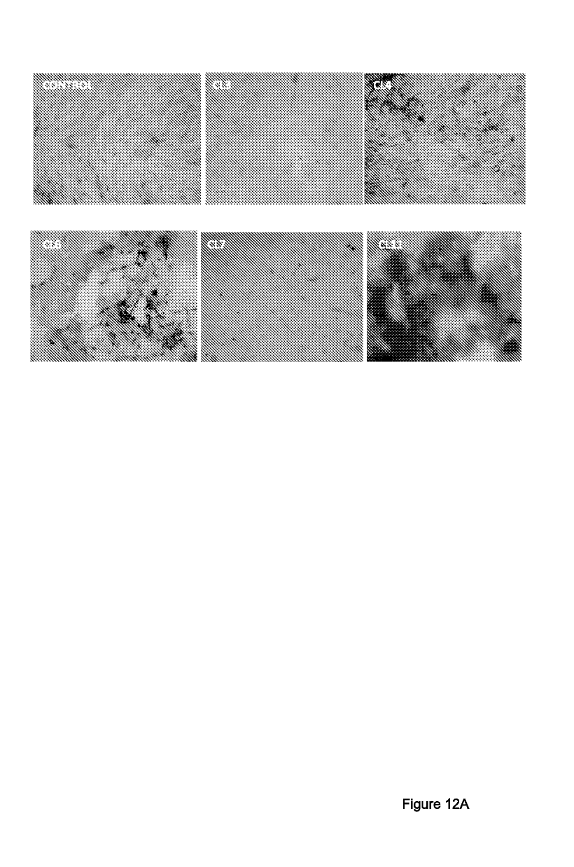

FIG. 13A-B fluorescent microscopy of hMSC on experimental materials utilising polymer substrates at 14 or 28 days

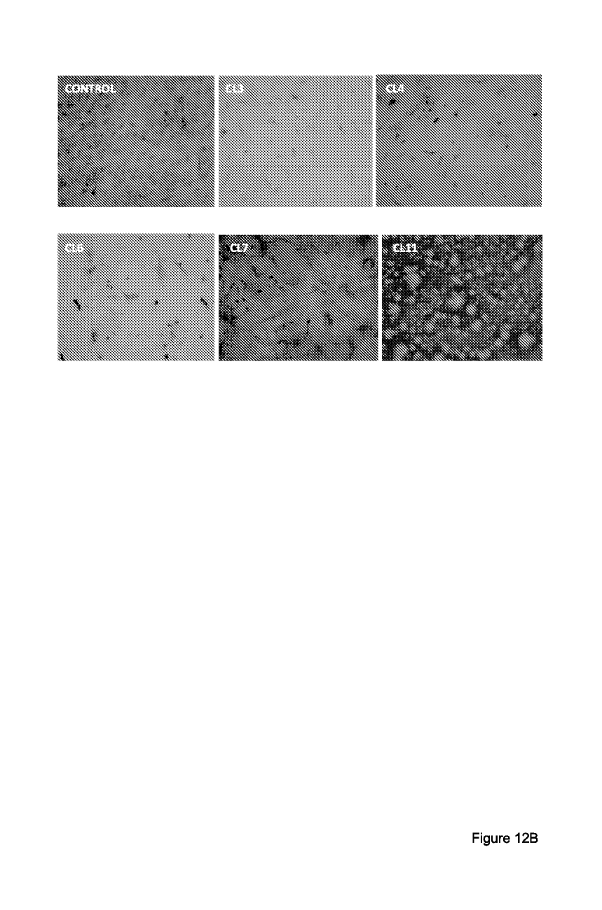

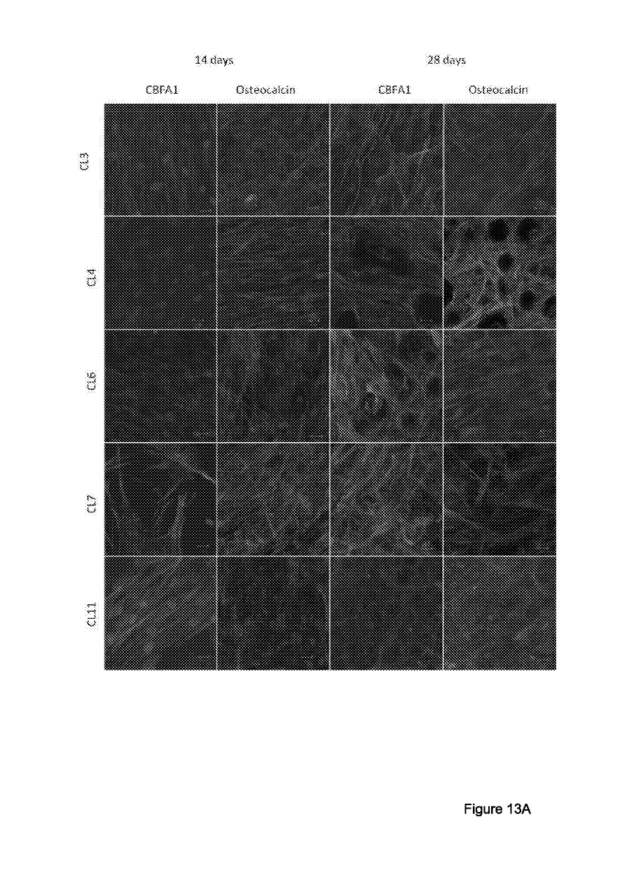

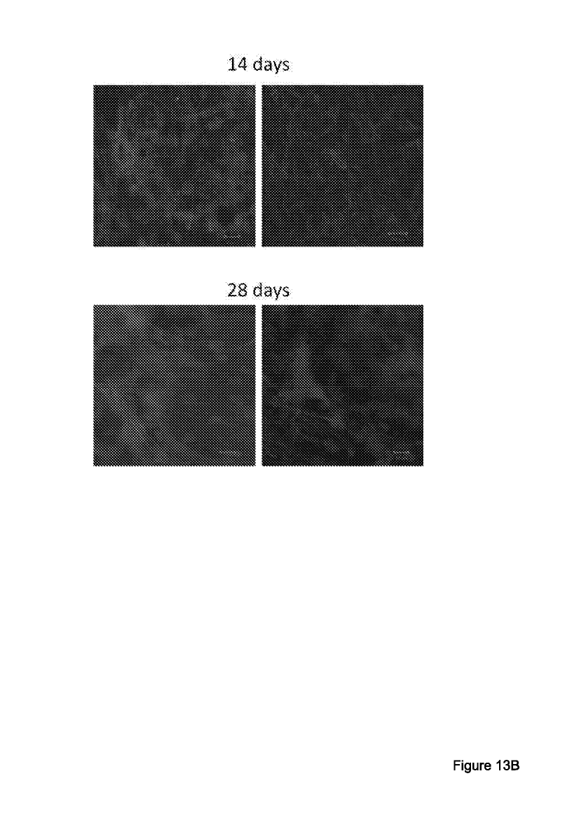

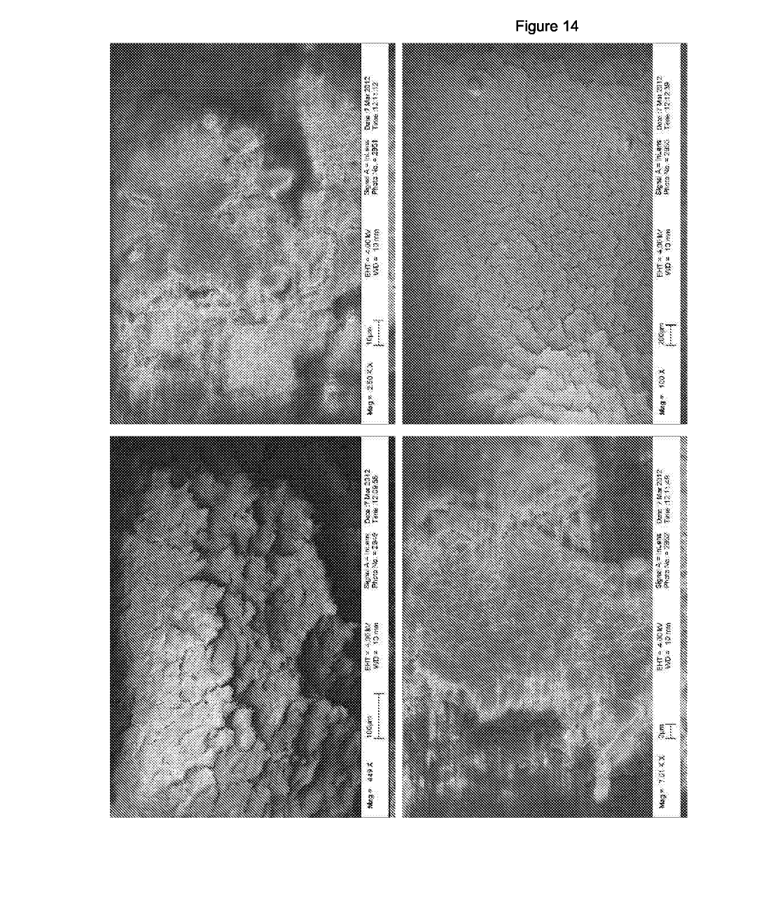

FIG. 14 shows scanning electron microscopy images of osteoblasts cultured on a control material for 7 days.

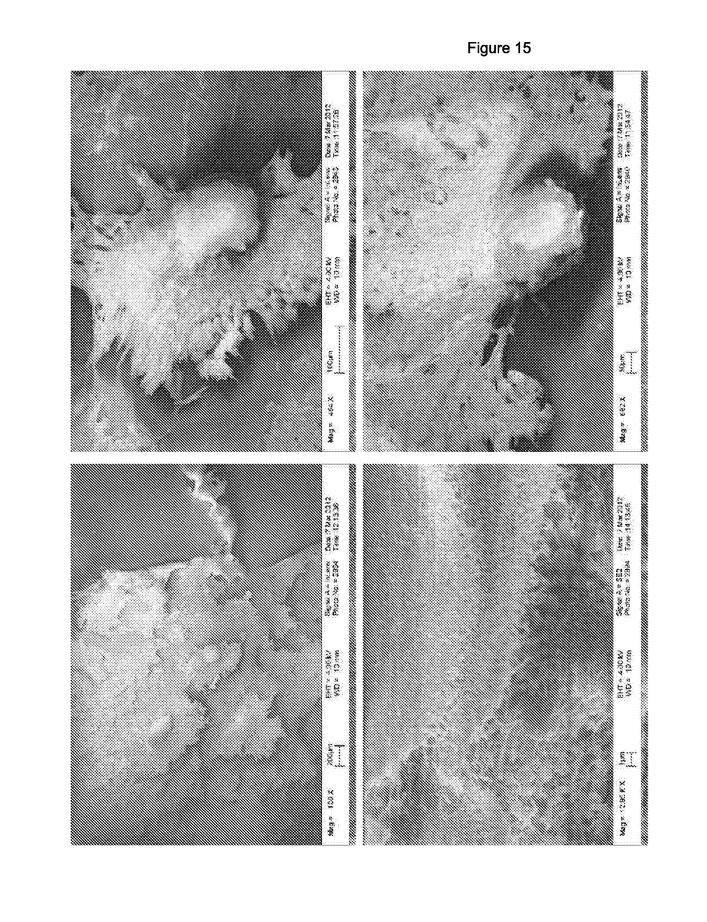

FIG. 15 shows scanning electron microscopy images of osteoblasts cultured on an experimental comparator material (CL3) for 7 days.

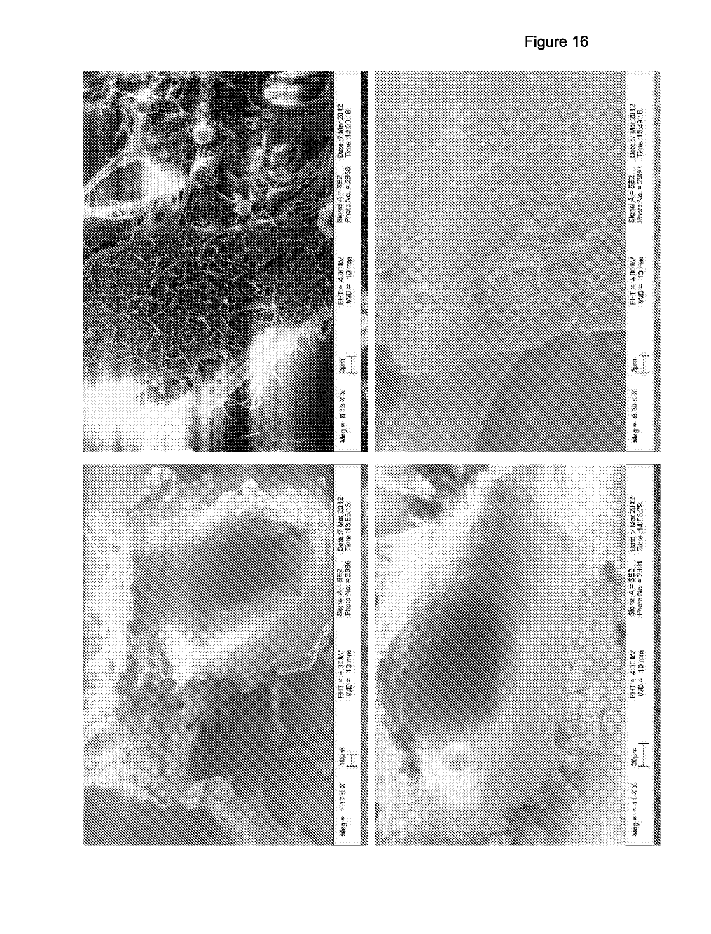

FIG. 16 shows scanning electron microscopy images of osteoblasts cultured on an experimental comparator material (CL4) for 7 days.

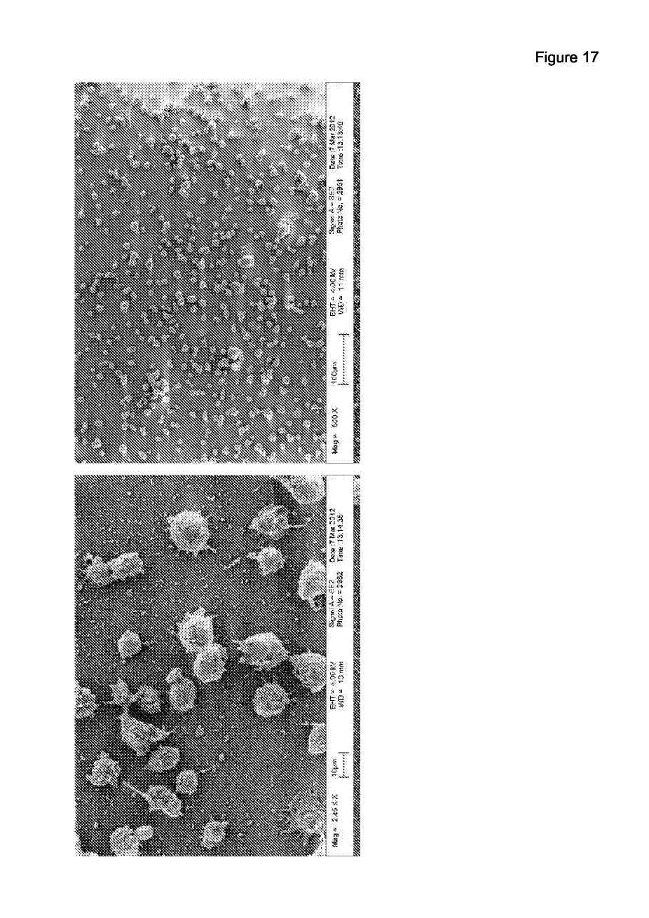

FIG. 17 shows scanning electron microscopy images of osteoblasts cultured on an experimental comparator material (CL6) for 7 days.

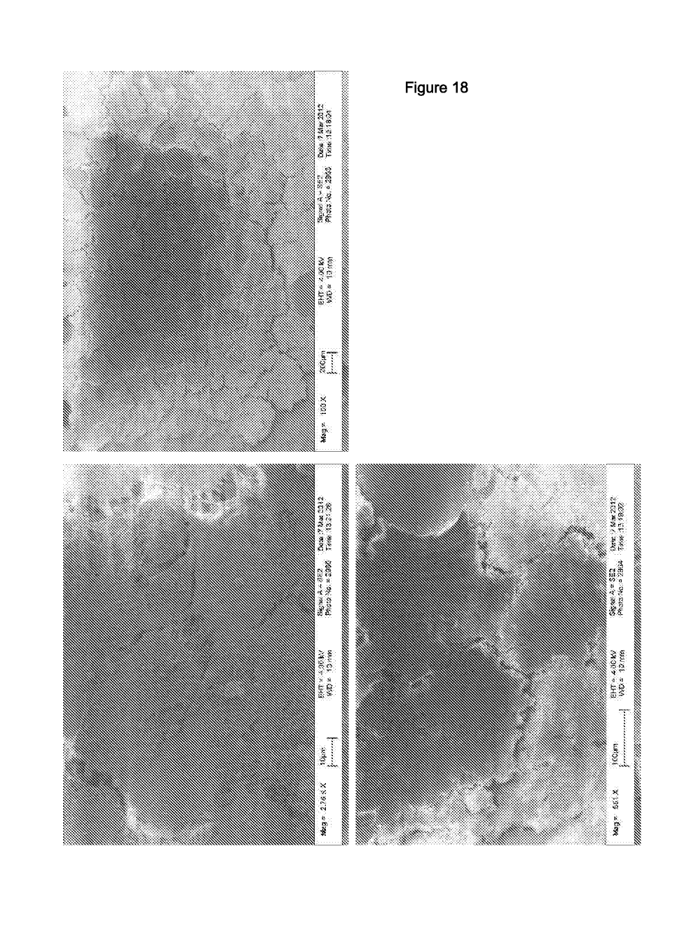

FIG. 18 shows scanning electron microscopy images of osteoblasts cultured on an experimental comparator material (CL7) for 7 days.

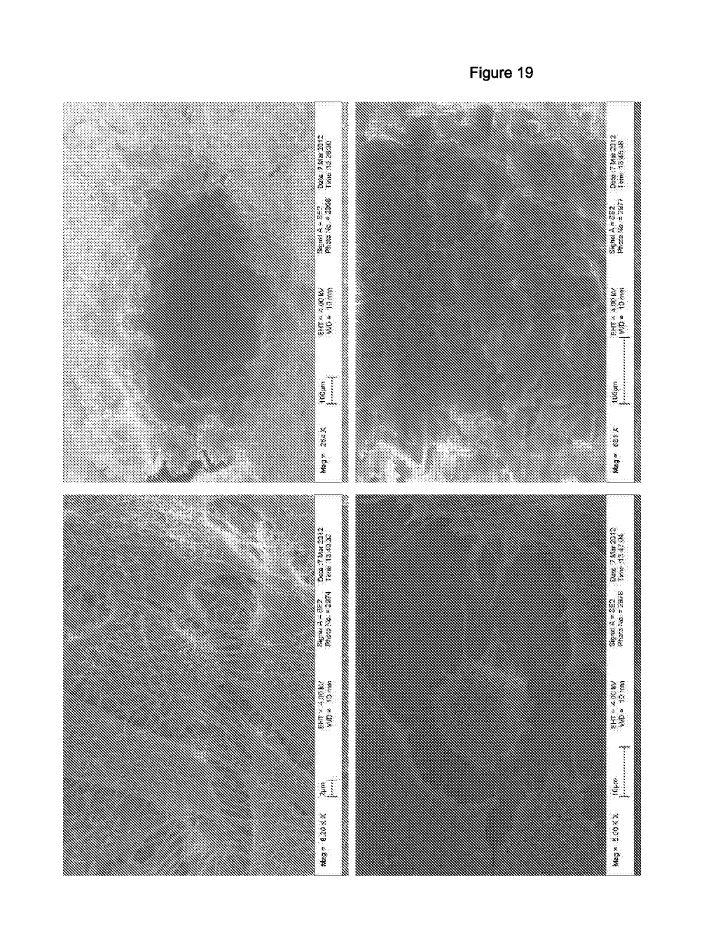

FIG. 19 shows scanning electron microscopy images of osteoblasts cultured on a material of the invention (CL11) for 7 days.

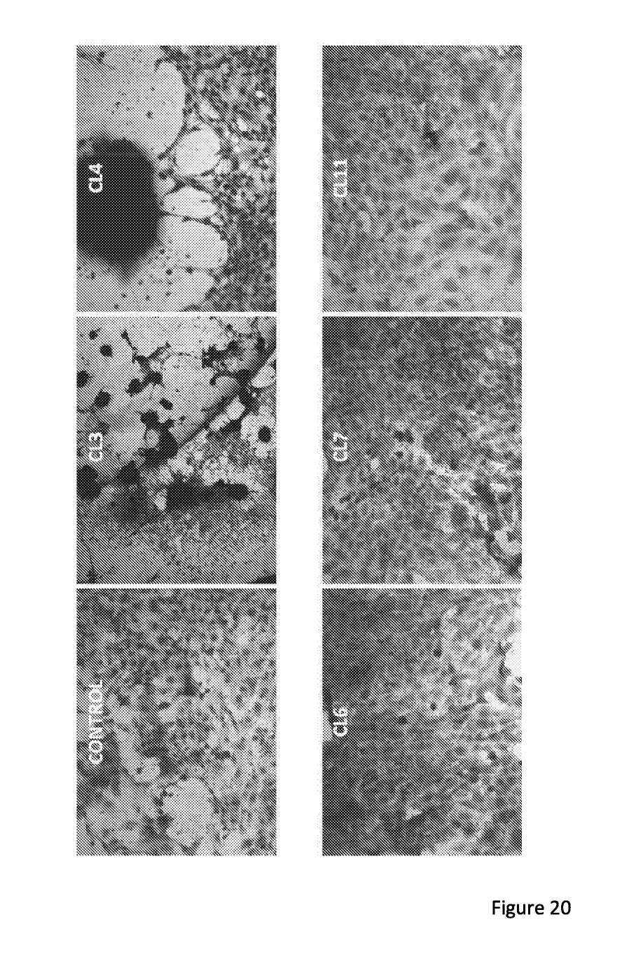

FIG. 20 compares micrographs showing von Kossa staining of osteoblast populations cultured on control or experimental comparator materials, or a material of the invention, for 7 days.

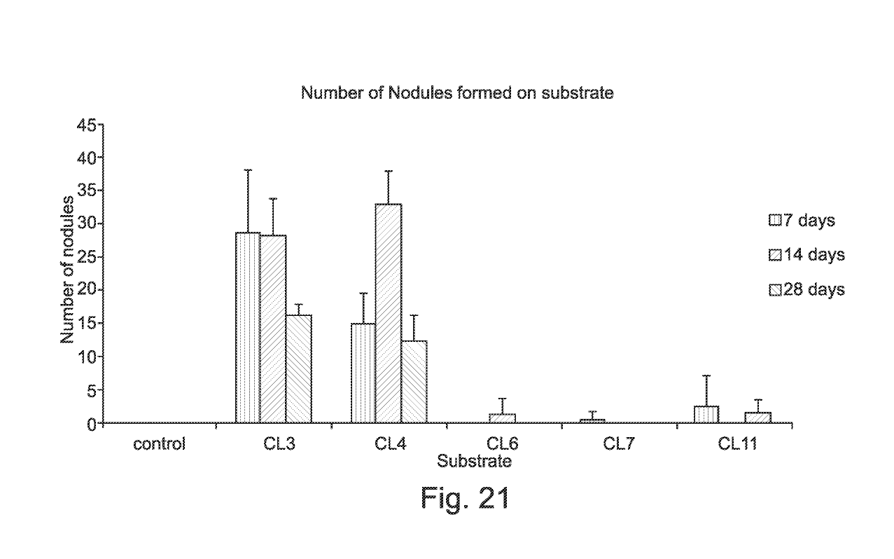

FIG. 21 is a graph comparing the formation of nodules among osteoblast populations cultured on control or experimental comparator materials, or a material of the invention, for 7, 14, or 28 days.

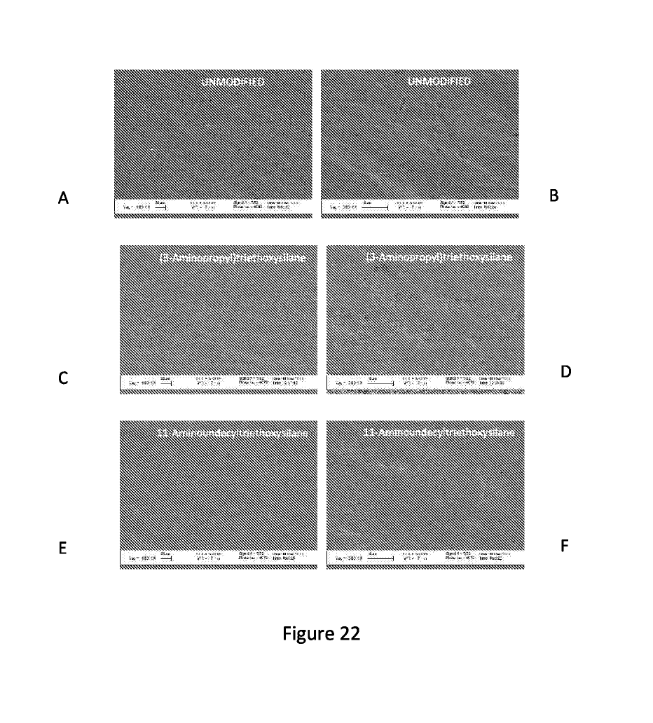

FIG. 22 shows scanning electron microscope (SEM) images of the surfaces of control materials (labelled “unmodified” in A and B), comparator materials (labelled “3(Aminopropyl)triethoxysilane” in C and D), and materials of the invention (labelled “11-Aminoundecyltriethoxysilane”) formed on titanium substrates at different magnifications (panels B, D, and F are at greater magnification than panels A, C, and E).

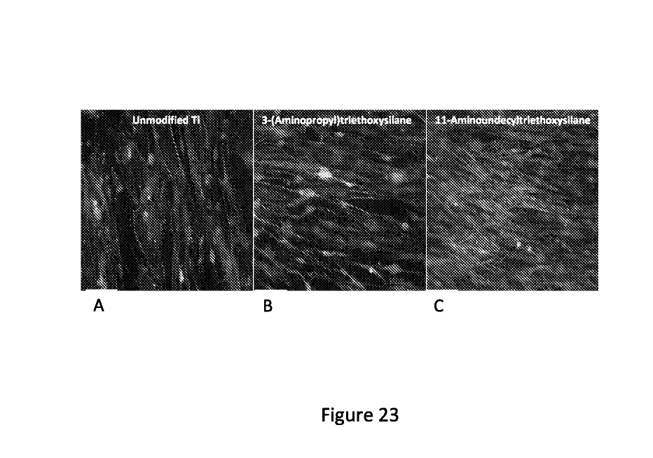

FIG. 23 shows photomicrographs of MSCs cultured for 28 days on titanium substrates either untreated (control material labelled “Unmodified Ti”), or treated with silanes to yield comparator materials (labelled “3-(Aminopropyl)triethoxysilane”) or osteoinductive materials of the invention (labelled “11-Aminoundecyltriethoxysilane”).

### EXPERIMENTAL RESULTS

Study I

1 Preparation of Osteoinductive Materials of the Invention and Experimental Comparison Materials

1.1 Preparation of Materials on Glass Substrates

Glass was cleaned using a 0.5M solution of sodium hydroxide (Sigma, UK) for 30 minutes in an ultrasonic bath, the samples were then washed in three changes of distilled water, and placed in 1M Nitric acid for 30 minutes in an ultrasonic bath. Samples were then washed with three changes of distilled water and dried in a 50° C. oven. Clean coverslips were then modified using the silanes in Table 1 in 0.1 M solutions for 30 minutes.

The coverslips modified using 11-aminoundecyltriethoxysilane represent osteogenic materials of the invention (and the manner in which they have been manufactured represents a method of the invention), while the coverslips modified using other carbon chain lengths (CL3, CL4, CL6, and CL7) constitute experimental comparison materials.

Samples were then washed with Isopropyl alcohol for 5 minutes and then washed with distilled water prior to use.

1.2 PLGA Film Production and Preparation of Materials on PLGA

Clean 12 mm glass cover slips were coated with chromium using Emtech 575× sputter coater (Emtech, UK), to provide a surface for the PLGA film to form hydrogen bonds with. 100 μl of 10% 85:15 PLGA (Sigma, UK) in chloroform (UOL chemical stores, UK) was spin coated onto the chromium coated cover slips using WS-400B-6NPP/LITE spin coater (Laurell Technologies Corporation, UK). Oxygen plasma was used to functionalize the polymer (Emtech, UK), at the previously optimised settings of 30 kW for 2 minutes.

As with the glass substrates described above, clean coverslips coated with PLGA were then modified using the silanes in Table 1 in 0.1M solutions for 30 minutes.

The coverslips modified using 11-aminoundecyltriethoxysilane represent osteogenic materials of the invention (and the manner in which they have been manufactured represents a method of the invention), while the coverslips modified using other carbon chain lengths (CL3, CL4, CL6, and CL7) constitute experimental comparison materials.

Samples were then washed with Isopropyl alcohol for 5 minutes and then washed with distilled water prior to use.

TABLE 1

| | |
|---|---|
| CL3 | (3-Aminopropyl)triethoxysilane (Sigma) |
| CL4 | 4-(triethoxyslyl(butan-1-amine (fluorochem) |
| CL6 | 3-(2-Aminoethylamino) |
| | Propyldimethoxymethylsilane (Sigma) |
| CL7 | N-(6Aminohexyl)amnomethyltriethoxysilane (fluorchem) |
| CL11 | 11-Aminoundecyltriethooxysilane (fluorochem) |

1.3 Von Kossa Stain

Samples were hydrated using 3 submersions in distilled water for 2 minutes each, then covered with 2% silver nitrate solution (Sigma UK) and placed under a UV lamp for 1 hour. Samples were washed with distilled water and put in a 2.5% sodium thiosulphate solution (Sigma UK) for 3 minutes. Samples were then washed with distilled water and counterstained using Harris's haematoxylin (Sigma UK) for 5 minutes, washed in running tap water for 5 minutes and differentiated in 1% acid alcohol for 2 seconds before being washed in distilled water. Samples were then dehydrated through increasing concentrations of alcohols (70%, 90% and 100%) for 2 minutes each and cleared in xylene (BDH, UK) for 2 minutes then mounted with glass coverslips in DPX (BDH, UK).

2 Analysis of Physical and Chemical Properties of the Experimental Materials

2.1 Atomic Force Microscopy (AFM) and Measurements

Images obtained via atomic force microscopy of experimental materials formed on glass or polymer substrates are respectively shown in FIGS. 1 and 2. Of these experimental materials, those designated CL11 represent materials of the invention.

It can be seen that the distribution of the hydrocarbyl chains and presenting amine groups is not uniform, over the surface of the materials, giving rise to features having a “wave-like” appearance when visualised by AFM. The uneven distribution is observed in respect of the various experimental materials, but distinctions can be made between the features found on materials of the invention, and those found on comparator or control materials.

Quantification of the maximum heights of the features on the surfaces of the experimental materials (as measured by AFM) is shown in FIGS. 3 and 4. Here it can be seen that the maximum feature height in materials of the invention is significantly larger than the maximum height achieved in control or comparator materials.

2.2 Scanning Electron Microscopy

The surfaces of control and experimental materials (including a material of the invention designated CL11) were investigated using scanning electron microscopy. As can be seen from the representative images shown in FIGS. 5 and 6, the features on the surface of the materials were below the size that can be resolved by scanning electron microscopy, confirming that the features are on a micrometre and nanometre scale, as illustrated in the AFM images.

On materials of the invention (the CL11 modified substrates) there is clear evidence of a network formation on both the glass and PLGA modified substrates, representative of the open honeycomb structure described in later sections. This was more apparent on materials of the invention using a PLGA polymer substrate, but enhanced contrast of the images of the materials of the invention based on glass substrates clearly shows the presence of this network structure on the surface. This is not observed on any of the other modified or control substrates, therefore is a distinguishing feature of the materials of the invention.

2.3 Water Contact Angle (WCA) Measurements

Double sided materials were used for this technique, produced in the same way as previously described. WCA were measured using a Camtel Dynamic Contact Angle (DCA) machine (Camtel LTD, UK). 6 replicates were carried out per repeat, a total of 4 repeats were carried out for each test substrate. Values provided are average+/−standard deviation. Statistical analysis was carried out using ANOVA Waller Duncan and Tukey models.

The results obtained with experimental materials using glass or polymer substrates are respectively set out in FIGS. 7 and 8. Here it can be observed that particularly in the results obtained from polymer-based materials (FIG. 8) the surface energy of the materials of the invention (CL11) is distinctly lower than the surface energy of other comparator materials.

2.4 X-ray Analysis of Glass (Control) and Modified Films

Dry modified films and glass coverslips were coated with carbon using a carbon coater (EMTECH, UK). X-ray microanalysis was performed using a Leo 1550 SEM (Zeiss, UK) with an INCA system (Oxford Instruments). Points of analysis were imaged at 5 locations on the film. From each of these fields 10 spectrums were generated at random. The resulting data was analysed using ANOVA, to determine any statistically significant difference between the elements on the scaffolds.

Phosphate deposition is relevant to its role in the formation of an osteoinductive matrix that favours bone formation. As can be seen from FIG. 9, phosphate absorption onto the surfaces of experimental materials was only observed in respect of the materials of the invention (CL11), and comparator materials incorporating six and seven carbon chains. However, while phosphate absorption was observed in respect of each of these materials, the extent of phosphate absorption, and range of conditions (particularly with respect to surrounding phosphate concentration) in which phosphate absorption occurs, was greater for the materials of the invention than for comparators.

3 Analysis of Biological Properties of the Experimental Materials

3.1 Mesenchymal Stem Cell Response

Mesenchymal stem cells were cultured on experimental materials (utilising glass or polymer substrates) for 28 days. The experimental materials included materials of the invention (CL11). Cells were investigated at various timepoints, and the results are shown in FIGS. 10 and 11.

Panels A and B of FIG. 10 respectively show von Kossa staining of human mesenchymal stem cells (hMSCs) cultured for 7 and 28 days on the various experimental and control materials (CL11 representing a material of the invention).

Positive von Kossa staining (indicating the presence of calcified extracellular matrix) is indicated by a dark brown/black colour. It can be seen in FIG. 10A that, after 7 days in culture, a denser matrix is observed on the CL11 sample than is found on the other control or comparator materials.

Turning now to FIG. 10B, von Kossa positive calcified extracellular matrix is once again stained with a dark brown/black colour. There is clear evidence of remodelling and reorganisation of the calcified extracellular matrix on the CL11 material (of the invention), indicating the osteoinductive properties of this material. In contrast, while the comparator material made with a six carbon chain also shows evidence of a calcified extracellular matrix, other indications suggest that this is not associated with efficient osteoinduction or formation of bone-like material.

Materials of the invention produced on a polymer substrate are able to induce a similar response in cultured hMSCs, as shown in panels A and B of FIG. 12. Indeed, the distinction between the effects observed in cultures grown on materials of the invention, and those grown on comparator or control materials, are even more pronounced in this case.

While cells adhere to and grow on the other materials (as shown by haematoxylin and eosin staining), deposition of calcified extracellular matrix is only observed in respect of the material of the invention (CL11). Here the pattern of the distribution of the matrix reflects the underlying topography of the features found exclusively in the material of the invention.

FIGS. 11 and 13 illustrate immunofluorescent labelling of hMSCs grown on a range of experimental materials (including a material of the invention designated CL11) utilising either glass (FIG. 11) or PLGA polymer (FIG. 13) substrates. In each case, images are shown for cells cultured for either 14 or 28 days on the materials. Cell nuclei are stained with DAPI (blue colour), and cells have also been labelled for the presence of the transcription factor CBFA1 (red colour in cell nuclei), cytoskeletal actin (green colour), and ostocalcin a major component of the osteogenic extracellular matrix (red colour).

The images set out in FIGS. 11 and 13 clearly show that only cells grown in contact with the materials of the invention (whether on glass or polymer substrates) form an osteocalcin rich matrix when cultured in vitro under basal conditions. While osteocalcin staining is found in cells grown on other materials, this has an intracellular location and so cannot be associated with the formation of an osteocalcin-rich extracellular matrix (which is a definitive marker of osteoinduction).

In contrast, the osteocalcin produced by cells grown on materials of the invention is deposited in a dense extracellular matrix. Even by day 7, the cells can be seen to be embedded within this matrix (see FIG. 11B), and at the cells subsequently reorganise and remodel this matrix. By 28 days this leads to the formation of cell-containing pits within the surface of the matrix, indicative of the cells undergoing a change from an osteoblast to osteocyte phenotype. Osteocalcin arrangements illustrating these changes can be seen in images taken after 14 or 28 days in culture (right hand columns of FIGS. 11A and 13A, FIGS. 11B-11E and 13B).

3.2 Human Osteoblast Response

Primary derived human osteoblasts were also cultured on experimental materials (including a material of the invention designated CL11) for various periods of time (up to 28 days). Scanning electron micrographs illustrating representative populations of these cultured cells after 7 days are set out in FIGS. 14 to 19, and von Kossa staining of 7 day cultures is shown in FIG. 20.

When cells were cultured in contact with CL11 materials (osteoinductive materials in accordance with the invention) there was no observable difference in the cellular response when compared to control, CL6 and CL7 substrates. All of these substrates supported osteoblast attachment and growth but did not result in the formation of calcified cell clusters as observed on CL3 and CL4 substrates. Therefore the observed osteoinductive effect associated with materials of the invention when used as a surface for stem cell growth did not result in the significant enhancement of osteoblast cell culture. This phenomenon i.e. enhanced calcified matrix and nodule production by osteoblasts was only associated with the specific material parameters produced by CL3 and CL4 modifications. Nodule formation was quantified using SEM and image analysis and the results are set out in FIG. 21.

Study II

4 Preparation of Further Controls, Comparators, and Osteoinductive Materials of the Invention

The data presented in Study I (above) illustrate that optimal —NH₂ surface modification parameters (silane modification of a base substrate with 11-Aminoundecyltriethoxysilane) enable to production of materials of the invention with enhanced osteoinductive properties on glass or tissue culture polystyrene (TOPS) substrates. The current Study confirms that the process of modification is translatable to metallic, titanium, substrates, resulting in the generation of osteoinductive materials of the invention that offer superior properties to comparator materials made using other —NH₂ modifications (employing hydrocarbyl groups with a length other than 10 to 12 carbon atoms) on the same substrates.

4.1 Methods

4.1.1 Material Modification

5.0 mm diameter polished titanium (Ti) discs were immersed into a 10% NaOH solution for 1 hour then washed thoroughly with high pure water and dried with nitrogen, then stored in a vacuum desiccator prior to introduction of amine groups (NH₂) on the surface by silanation. To introduce the —NH₂ Ti discs were dipped into 2% silane 1 (3-Aminopropyl)triethoxysilane) or silane 2 (11-Aminoundecyltriethoxysilane) isopropyl alcohol solution (95% in water) at 37° C. for 1 hour. Those discs dipped in 3-Aminopropyltriethoxysilane yielded a comparator material that is not of the present invention, while the discs dipped in 11-Aminoundecyltriethoxysilane yielded osteoinductive materials of the invention.

The titanium discs were then rinsed twice with isopropyl alcohol, ultra-pure water and ethanol and then dried at 135° C. for half an hour and stored in a vacuum desiccator.

4.2 Surface Stability Study

The shelf life of medical devices is a crucially important factor for the healthcare provider and the manufacturer. To study the stability of the coatings, the same surface modification strategy has been used to modify coverslips. The modified coverslips were stored in dry air, water for 1, 2 and 4 weeks for investigating the stability of the coating.

Two methods have been used to sterilize the silane modified coverslips for studying their effect on the stability of the coating. The first method is to sterilize the coverslips by 70% ethanol for 15 mins; the other is to autoclave the coverslips at 121° C. for 15 mins.

4.3 Materials Characterization

4.3.1 Scanning Electronic Microscopy (SEM)

SEM was performed using a Leo/Zeiss 1550 Field Emission Scanning Electron Microscope. Briefly, one titanium disc from each group (control, comparator, or material of the invention) was mounted onto an aluminium stub using double sided adhesive carbon tape and sputter coated with chromium using an EMITECH K575X coater. The surface micro-structures and profiles were observed using Field Emission Scanning Electron Microscopy (FE-SEM) (LEO 1550, Cambridge, UK). The coated sample was placed in the vacuum chamber of the SEM and viewed at a voltage of 5 kV.

4.3.2 X-Ray Element Analysis

X-ray analysis was performed using Oxford Instruments INCA software version 4.15. The system was calibrated using a MAC (Micro Analysis Consultants Ltd) Standard, number 5383, using the unmodified (control) titanium sample. 5 areas of the sample surface were chosen at random, at magnification ×1000, and the area scanned for 5 mins using the ANALYZER function.

4.3.3 Contact Angle

Dynamic contact angles of the coverslips samples with or without modification in deionised purified water were analysed using the Wilhelmy contact angle test. Briefly, the contact angles for all the samples were determined using a Dynamic Contact Angle Tensiometer (CDCA 100, Camtel Ltd., Royston, Herts, UK) at 22±0.5° C. Each sample was immersed into, and retracted from the wetting solution (deionised pure water) at a rate of 0.060 mm/s. The wetting force at the solid/liquid/vapour interface was recorded via the electrobalance as a function of both time and immersion depth, and was converted into advancing contact angles. The values reported for dynamic advancing angles of are mean and standard deviations of four measurements.

5 Results

5.1 Surface Modification of Ti Discs

Scanning electron microscopy images illustrating the surfaces of the control (“unmodified”), comparator materials (“(3-Aminopropyl)triethoxysilane”), and materials of the invention (“11-Aminoundecyltriethoxysilane”) are shown in FIG. 22.

The results of surface element analysis results of titanium discs before and after amine silane modification are set out in Table 2.

TABLE 2
Elements composition of Ti discs
surface with/without modification

| Element | | 3-Aminopropyl- | 11-Aminoundecyl- |
|---|---|---|---|
| (atomic %) | Ti discs | triethoxysilane | triethoxysilane |
| C | 0.00 ± 0.26 | 11.96 ± 0.62 | 15.59 ± 2.00 |
| Si | 2.14 ± 0.26 | 1.56 ± 0.03 | 1.59 ± 0.06 |
| Ti | 97.86 ± 0.26 | 86.36 ± 0.79 | 82.78 ± 2.00 |

The SEM images show that after silane modification, the surface of titanium discs became smoother, especially after modification by silane with long methylene chains (i.e. in the osteoinductive materials of the invention modified with 11-Aminoundecyltriethoxysilane). Element analysis result shows carbon increases and titanium decreases after silane modification. These results indicated that the surfaces of treated titanium discs have been successfully modified with amine terminated silanes.

5.2 Surface Modification Stability

To study the stability of the silane modification, the surface contact angles were measured with glass coverslips which have been untreated, or modified with the same strategy (treatment with either 3-Aminopropyltriethoxysilane to produce a comparator material, or 11-Aminoundecyltriethoxysilane to produce an osteoinductive material of the invention).

TABLE 3
Contact angles results of coverslips under different
sterilization and storage conditions

| Contact angles | | Unmodified | 3-Aminopropyl- | 11-Aminoundecyl- |
|---|---|---|---|---|
| (°) | | Coverslips | triethoxysilane | triethoxysilane |
| | | 49.90 ± 3.66 | 75.45 ± 1.63 | 91.39 ± 1.41 |
| Autoclave | | 47.37 ± 5.79 | 71.51 ± 3.11 | 89.55 ± 4.40 |
| (121° C.) | | | | |
| Ethanol (70%) | | 49.22 ± 4.31 | 73.18 ± 4.95 | 88.73 ± 3.31 |
| Water | 1 week | — | 67.19 ± 1.76 | 82.02 ± 2.69 |
| | 2 week | — | 67.34 ± 3.00 | 85.05 ± 3.45 |
| | 4 week | — | 66.94 ± 2.13 | 81.54 ± 7.74 |
| Air | 1 week | — | 77.86 ± 8.76 | 90.21 ± 5.79 |
| | 2 week | — | 75.49 ± 2.95 | 92.33 ± 5.15 |
| | 4 week | — | 72.61 ± 4.83 | 89.36 ± 7.33 |

The results set out in Table 3 shows that sterilisation utilising an autoclave (standard 121° C. sterilisation) and 70% ethanol sterilisation, did not affect the overall hydrophobic/hydrophilic properties of the material. Slight changes in contact angle were within acceptable limits and did not affect the overall properties of the surface. The result indicate that osteoinductive materials of the invention, as exemplified by the silane modified titanium discs, can be sterilized by autoclave at 121° C. or with 70% ethanol with no detrimental effect to the properties of the modified surfaces.

The result in Table 3 also shows that the silane modifications of coverslips are stable in water or dry air at least for 4 weeks.

6 Analysis of the Osteoinductive Properties of the Materials

As with previous cell testing detailed in Study I above, modified and unmodified titanium discs were cultured in contact with commercially available well characterised human mesenchymal stem cells (Lonza), in basal medium. The ability of the materials to induce osteogenic differentiation was characterised qualitatively using markers for collagen I (major component of extracellular matrix) and osteocalcin (marker of osteogenic differentiation).

Photomicrographs of cells grown on control materials (“unmodified”), comparator materials (“3-(Aminopropyl)triethoxysilane”), and osteoinductive materials of the invention (“11-Aminoundecyltriethoxysilane”) are shown in FIG. 23. The images are of MSCs cultured in contact with the various materials utilising titanium substrates for 28 days and stained for osteocalcin (red), F-actin (marker of cellular cytoskeleton, green) and cell nuclei (blue). Cells cultured in contact with the titanium discs treated with 11-Aminoundecyltriethoxysilane to produce materials of the invention demonstrated a greater level of osteocalcin expression compared to un-modified (control) and comparator substrates modified with 3-(Aminopropyl)triethoxysilane).

Data retrieved from the study proved that cells cultured in contact with 11-Aminoundecyltriethoxysilane in materials of the invention showed a greater level of osteogenic activity compared to other amine modified and unmodified titanium substrates. This data is in line with previously provided data regarding the optimal osteogenic properties of amine modified glass and PLGA.

1. An osteoinductive material comprising:
a substrate; and
a layer of a hydrocarbyl group comprising 10 to 12 carbon atoms, the hydrocarbyl group having one end attached to the substrate and a presenting amine (—NH2) group at the unattached end.

2. An osteoinductive material according to claim 1, wherein the hydrocarbyl group is straight.

3. An osteoinductive material according to claim 1, wherein the hydrocarbyl group is an 11 carbon alkyl chain.

4. An osteoinductive material according to claim 1, wherein the wherein the hydrocarbon is attached to the substrate via a Si—O—Si bond.

5. An osteoinductive material according to claim 1, wherein the hydrocarbyl group is substantially the only hydrocarbon present on a hydrocarbyl group-coated surface of the osteoinductive material.

6. An osteoinductive material according to claim 1, comprising multiple layers of the hydrocarbyl group and presenting amine.

7. An osteoinductive material according to claim 6, wherein the multiple layers are distributed at different depths over the surface of the material.

8. An osteoinductive material according to claim 7, wherein the multiple layers provide nanometre-scale features on the surface of the osteoinductive material.

9. An osteoinductive material according to claim 8, wherein the maximum feature height is at least 40 nm.

10. An osteoinductive material according to claim 8, wherein the maximum feature height is between approximately 50 and 155 nm.

11. An osteoinductive material according to claim 8, wherein the maximum feature height is at least 60 nm.

12. An osteoinductive material according to claim 8, wherein the maximum feature height is at least 90 nm.

13. An osteoinductive material according to claim 8, wherein the maximum feature height is between approximately 90 and 150 nm.

14. An osteoinductive material according to claim 8, wherein the substrate comprises a polymer and the maximum feature height is approximately 100 nm.

15. An osteoinductive material according to claim 8, wherein the substrate comprises glass and the maximum feature height is approximately 140 nm.

16. An osteoinductive material according to claim 8, wherein the features have an open honeycomb arrangement, when viewed microscopically.

17. An osteoinductive material according to claim 1, wherein the water contact angle at the surface of the material is less than 100°.

18. An osteoinductive material according to claim 17, wherein the water contact angle at the surface of the material is between approximately 85° and 95°.

19. An osteoinductive material according to claim 18, wherein the water contact angle at the surface of the material is approximately 90°.

20. An osteoinductive material according to claim 1, wherein the substrate comprises one or more constituents selected from the group consisting of: glass, metals; polymers; ceramics; plastics; and hydroxyapatite.

21. An osteoinductive material according to claim 20, wherein the substrate comprises a metal selected from the group consisting of: titanium; titanium alloys; cobalt chrome; and
steel.

22. An osteoinductive material according to claim 1, wherein the substrate is in the form of a medical implant, or part thereof.

23. An osteoinductive material according to claim 22, wherein the substrate is in the form of an orthopaedic implant or part thereof.

24. An osteoinductive material according to claim 22, wherein the substrate is in the form of a dental implant or part thereof.

25. An osteoinductive material according to claim 1, wherein the substrate is in the form of a cell culture vessel.

26. An osteoinductive material according to claim 25, wherein the cell culture vessel is selected from the group consisting of: cell culture dishes (such as Petri dishes); cell culture flasks; cell culture plates; cell culture bottles (such as roller bottles); cell culture bags; and cell culture beads.

27. A method of producing an osteoinductive material, the method comprising contacting a surface of a substrate with a solution containing a compound comprising a hydrocarbyl group comprising 10 to 12 carbon atoms and a terminal amine group, such that a coating of the hydrocarbyl group is formed on at least a portion of a surface of the substrate.

28. A method according to claim 27, wherein the hydrocarbyl group is 11 carbons long.

29. A method according to claim 28, wherein the hydrocarbyl group is provided in the form of 11-aminoundecyltriethoxysilane.

30. A method according to claim 27, wherein the compound comprising the hydrocarbyl group is provided at a concentration of approximately 0.1 M.

31. A method according to claim 27, wherein the surface of the substrate is contacted with the solution by immersion of the substrate within the solution.

32. A method according to claim 27, wherein the surface of the substrate is contacted with the solution by spraying the solution onto the surface of the substrate

33. A method according to claim 27, further comprising washing the coated substrate in alcohol and/or water.

34. A method according to claim 27, further comprising drying the surface of the substrate.

35. A method according to claim 27, wherein the substrate comprises one or more constituents selected from the group consisting of: glass, metals; polymers; ceramics; plastics; and hydroxyapatite.

36. A method according to claim 27, wherein the substrate comprises a metal selected from the group consisting of: titanium; titanium alloys; cobalt chrome; and steel.

37. A method according to claim 27, wherein the substrate is in the form of a medical implant, or part thereof.

38. A method according to claim 27, wherein the substrate is in the form of an orthopaedic implant or part thereof.

39. A method according to claim 27, wherein the substrate is in the form of a dental implant or part thereof.

40. A method according to claim 27, wherein the substrate is in the form of a cell culture vessel.

41. A method according to claim 40, wherein the cell culture vessel is selected from the group consisting of: cell culture dishes (such as Petri dishes); cell culture flasks; cell culture plates; cell culture bottles (such as roller bottles); cell culture bags; and cell culture beads.

42. A method according to claim 27, wherein the osteoinductive material is as defined in claim 1.

43. (canceled)

44. A method of producing bone or a bone precursor, the method comprising:
providing an osteoinductive material according to claim 1;
providing a population of cells with osteogenic potential; and
maintaining the cells in contact with the material until bone or a bone precursor is produced.

45. A method according to claim 44, wherein the cells with osteogenic potential are stem cells.

46. A method according to claim 45, wherein the stem cells are selected from the group consisting of: embryonic stem cells; cord blood stem cells; adult stem cells (including haematopoietic stem cells; mesenchymal stem cells; and dental pulp stem cells); and induced pluripotent stem cells.

47. A method according to claim 46, wherein the stem cells are mesenchymal stem cells.

48. A method according to claim 44, further comprising maintaining the cells in contact with the material until the bone precursor is remodelled into bone or a bone-like substance.

49. A method according to claim 44, wherein the cells are maintained in serum-free cell culture medium.

50. A method according to claim 44, wherein the cells are maintained in cell culture medium without osteogenic growth factors.

51. A method according to claim 50, wherein the cells are maintained in cell culture medium substantially free of exogenous growth factors.

52. A method of promoting integration of a medical implant, the method comprising providing to a subject in need of such treatment a medical implant comprising an osteoinductive material according to claim 1.

53. A method of producing bone or a bone precursor, the method comprising:
providing an osteoinductive material produced by a method according to claim 27;
providing a population of cells with osteogenic potential; and
maintaining the cells in contact with the material until bone or a bone precursor is produced.

54. A method of promoting integration of a medical implant, the method comprising providing to a subject in need of such treatment a medical implant comprising an osteoinductive material produced by a method according to claim 27.
